(19) United States
(12) Patent Application Publication
(10) Pub. No.: US 2007/0258850 A1
(43) Pub. Date: Nov. 8, 2007

(54) Heat Transfer System for Retorts and Method Therefore

(76) Inventors: Leonce J. Miller III, Mandeville, LA (US); Chris G. Greve, Covington, LA (US)

Correspondence Address: THOMPSON HINE L.L.P.;Intellectual Property Group, P.O. BOX 8801, DAYTON, OH, 45401-8801, US

(21) Appl. No.: 11/745,720

(22) Filed: May 8, 2007

Related U.S. Application Data
(60) Provisional application No. 60/798,988, filed on May 8, 2006.
(60) Provisional application No. 60/798,980, filed on May 8, 2006.

(51) Int. Cl. A61L 2/00; A61L 2/04

(52) U.S. Cl. 422/20; 422/307; 426/237

(57) ABSTRACT

A retort system includes a vessel including a crate receiving volume. A crate is located in the crate receiving volume, the crate including sides defining a product receiving volume. A plurality of tubes extend between opposite sides and through the product receiving volume. The tubes include nozzles distributed along their lengths for ejecting a heated fluid during a heating operation within the retort.

### CROSS-REFERENCE TO RELATED APPLICATIONS

This application claims priority to U.S. Provisional Application No. 60/798,988, entitled “Heat Transfer System for Retorts or the Like, and Method Therefore,” filed May 8, 2006, and U.S. Provisional Application No. 60/798,980, entitled “Sonic System for Enhancing Heat Transfer in a Retort or the Like,” filed May 8, 2006, the contents of both of which are hereby incorporated by reference as if fully set forth herein.

### TECHNICAL FIELD

The present invention relates to a method and apparatus for thermally treating foods, pharmaceuticals, and other products, and particularly to a system for enhancing heat transfer in a thermal treatment process (which may include sterilization, cooking, etc) involving a retort, autoclave, sterilizer or the like.

### BACKGROUND

To sterilize many foods, pharmaceuticals and other products, to make them “shelf-stable,” these products must be subjected to a sterilization method by heating the food, in its sealed container to a predetermined temperature. The product is held at this temperature for a product specific duration. This process is commonly referred to as an autoclave process, retort process or a sterilization process.

Prior patents have contemplated vibrating, shaking, rotating, or repositioning the product within the retort during the sterilization procedure, but in large batches of product which have been densely loaded in baskets, trays, racks, and such, there exists a problem providing even thermal distribution to all product during the treatment process.

Particularly, with baskets in an immersion-type retort which are subject to extreme agitation so as to lessen the batch time, the product situated along the outer periphery of the batch may receive more thermal energy from the surrounding fluid than the product in the core of the batch, resulting in disproportionate thermal transfer. This is because the fluid surrounding the batch is thermally affected as it contacts the outer product forming the batch, and is further affected as it migrates to the core of the product.

### SUMMARY

In an aspect, a retort system includes a vessel including a crate receiving volume. A crate is located in the crate receiving volume, the crate including sides defining a product receiving volume. A plurality of tubes extend between opposite sides and through the product receiving volume. The tubes include nozzles distributed along their lengths for ejecting a heated fluid during a heating operation within the retort.

In another aspect, a crate is sized and configured for placement within a retort. The crate is capable of holding product for a heating process within the retort. The crate includes sides defining a product receiving volume. A plurality of tubes extend between opposite sides and through the product receiving volume. The tubes include nozzles distributed along their lengths for ejecting a heated fluid during a heating operation within the retort.

In another aspect, a method of processing products in a retort system is provided. The method includes locating a crate within a crate receiving volume of a vessel. Products are heated within a product receiving volume of the crate by delivering heated fluid through a plurality of tubes extending between opposite sides of the crate and through the product receiving volume. The tubes include nozzles distributed along their lengths ejecting a heated fluid during a heating operation within the retort.

In yet another aspect, a method of thermally treating a product within a retort is provided. The method includes, placing the product within a vessel of the retort. A heat transfer medium is placed in contact with the product. Sonic or ultrasonic energy is delivered to the product through the heat transfer medium.

The present system can provide a means of evenly distributing the heat transfer fluid throughout the basket, from its core to the outer area, uniformly. Such a system can speed up heat transfer in an immersion retort by minimizing heat loss as it migrates toward the center of the batch, even in densely packed batches. The system may utilize conic or ultrasonic energy within a retort enclosure during a sterilization process, which can enhance the efficiency of heat transfer between a fluid transfer medium and product to be treated.

### BRIEF DESCRIPTION OF THE DRAWINGS

FIG. 1 is an isometric, side view of a stack of retort trays or baskets forming a crate;

FIG. 2 is an upper isometric, end, side and bottom view of a retort tray used to form the crate of FIG. 1;

FIG. 3 is a close-up, side, isometric view of the retort tray of FIG. 2;

FIG. 4 is a front view of an embodiment of a retort with the cube of FIG. 1 located therein;

FIG. 5 illustrates another embodiment of a crate; and

FIG. 6 illustrates another embodiment of a retort.

### DETAILED DESCRIPTION

The exemplary embodiments described herein provide an array of fluid distribution tubes or plenums which have a hollow core running through their length, as well as apertures or nozzles situated along their length, for dispersing the heat transfer fluid, for example, water or steam, evenly throughout the batch.

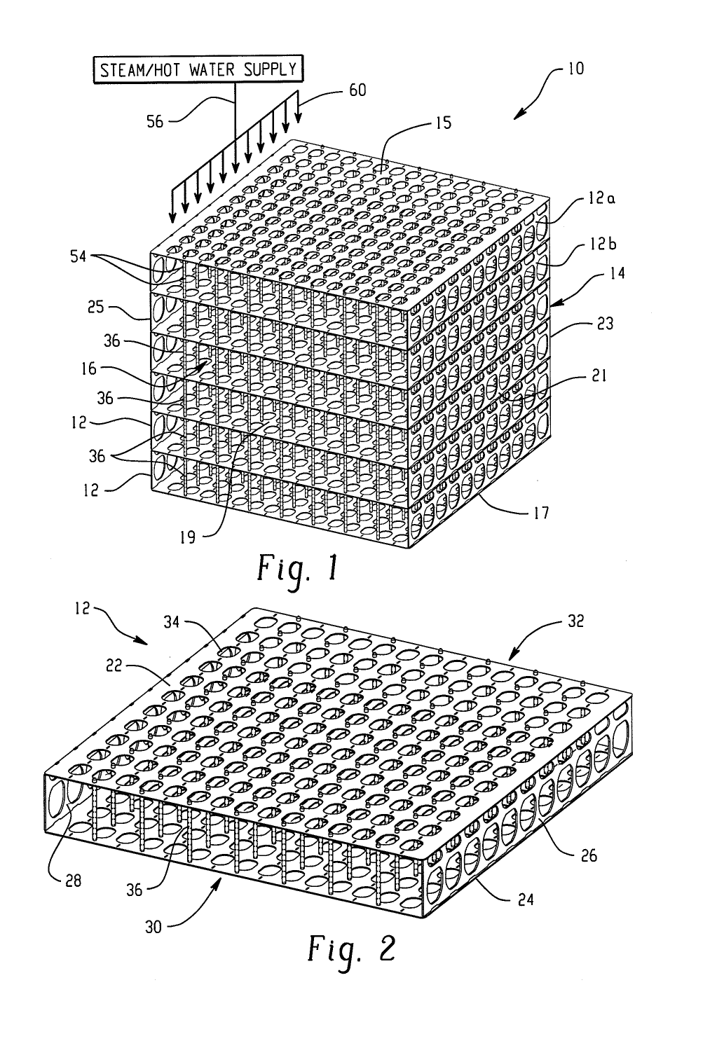

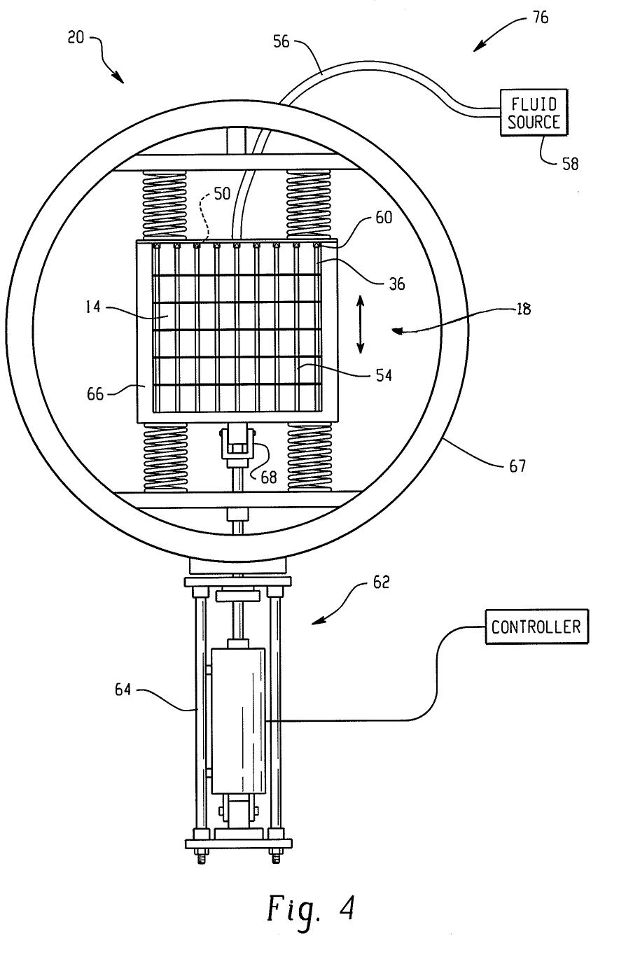

Referring to the FIG. 1, a stack 10 of trays 12 (also referred to as racks or baskets) form a cube 14 (also referred to as a crate) having a top 15, a bottom 17 and sides 19, 21, 23, 25. As can be seen, the trays 12 are stacked, one on top of the other to form the cube 14. The trays 12 include product receiving volumes 16 that are sized to receive product to be heated. The cube 14, once formed, is sized to be placed within a crate receiving volume 18 of a retort 20 (FIG. 4). In the illustrated embodiment, six trays 12 form the cube 14. However, more or less trays 12 may be used depending on, for example, the size of the crate receiving volume 18 of the retort 20.

Referring now to FIG. 2, each tray 12 is separable from an adjacent tray and includes a top wall 22, a bottom wall 24, side walls 26 and 28 and open ends 30 and 32. An array of openings 34 are formed in the top, bottom and side walls 22, 24, 26 and 28, respectively, and distributed about the periphery of the tray 12. The openings 34 facilitate steam or hot water distribution through the assembled crate 14.

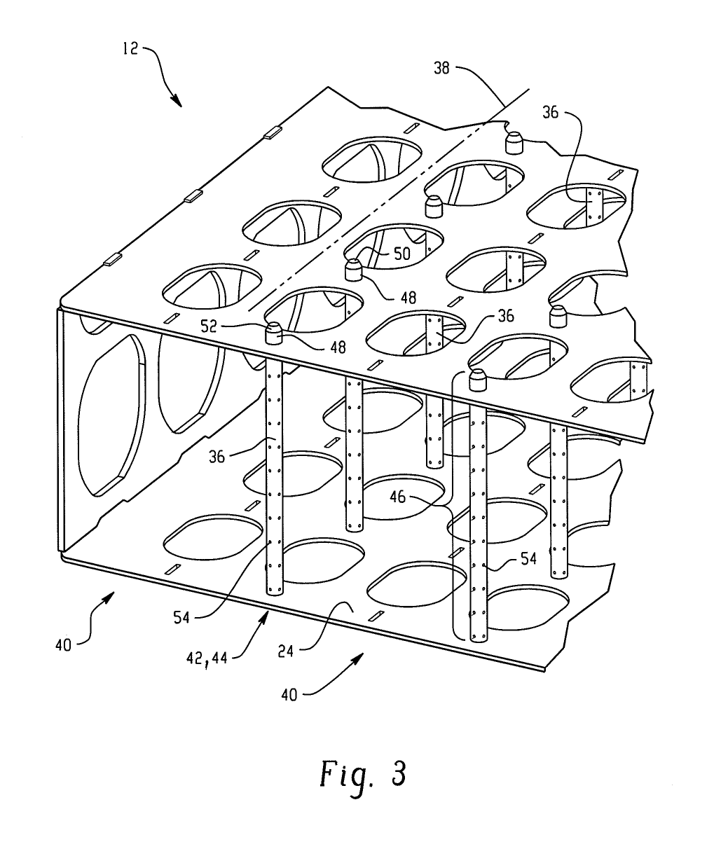

FIG. 3 shows an enlarged view of the tray 12. The tray 12 includes tube segments 36 that, in some embodiments, are evenly spaced and longitudinally aligned to form an imaginary line 38 between each row 40. Each line 38 of longitudinally aligned tube segments 36 is spaced apart from an adjacent line of tube segments to allow for receiving product in the tray 12. The line 38 of longitudinally aligned tube segments 36 also segregates each row 40 of product from an adjacent row.

Each tube segment 36 has a bottom end 42 with an opening 44 at the bottom wall 24 of each rack and a length 46 that extends between the top wall 22 and the bottom wall 24. The tube segments 36 further include an extended portion 48 that extends beyond the top wall 22 having a top 50, in some embodiments, including a tapered portion 52. An array of nozzles 54 are distributed along the length 46 of the tube segment 36. In some embodiments, the nozzles 54 are sized to eject steam or heated water at a desired rate. The nozzles 54 may be simple openings, or more complex structures.

The extended portion 48 of the tube segment 36 is sized to slidingly engage, in fluid impermeable fashion, the bottom end 42 of the tube segment 36 of an adjacent tray 12 stacked thereon (see trays 12a and 12b of FIG. 1). The stacked trays 12 form plural, vertically aligned and connected tube segments 36, acting effectively as a unitary ductwork of assembled tubes 54 (FIG. 1) for the distribution of fluid throughout the stack 10. The tapered portions 52 can facilitate a connection between the extended portions 48 and bottom ends 42 of tube segments 36 of stacked trays 12. In some embodiments, the extended portion 48 and/or bottom end 42 is provided with a gasket (not shown) to facilitate a fluid-tight seal.

Alternatively, instead of comprising stacked tube segments, each tube 54 may be a unitary, unsegmented perforated tube having a length of about the height of the cube 14 and installed therein as a unit through passages situated through the trays 12. In such a case, an end of each such tube could interface with a manifold or other fluid distribution system to receive the working fluid for the system.

Any suitable method may be used to form the trays 12. As one example, multiple plates of, for example, stainless steel may be stamped to form the openings 34 and welded together to form its sides. The tube segments 36 may also be formed of stainless steel and by extruding tubular forms and welding them within the trays as shown.

The above-described system may be used with the basket or cube loading system of U.S. Pat. No. 6,739,108, the contents of which are incorporated herein by reference, wherein trays are stacked layer by layer into a basket frame.

Referring now to FIG. 4, the crate 14 of stacked trays 12 is shown within the retort 20. The retort 20 includes a fluid delivery line or lines 56 that are used to deliver pressurized fluid from a source 58. Delivery nozzles or connection sockets 60 are in communication with the delivery line 56 and, when the trays are loaded into the retort for a processing operation, are connected to openings in the top 50 of the uppermost tube segments 36 (see also FIG. 1 where the nozzles/sockets 60 and line 56 are represented by arrows). In one example, the trays 12 are loaded into the retort 20, aligned and moved upward such that the nozzles/sockets 60 engage the upper ends of the tube segments 36.

In some embodiments, the basket or cube 14 can be modified to provide a manifold along its upper portion to engage the tapered top 50 of the tube segments 36 emanating from the uppermost rack 12 of the cube, the manifold further having a connection such as a socket connection, with a fluid distribution system within the retort once loaded therein, so as to provide fluid distribution through the tube segments. Further, the bottom end 42 of the tube segments 36 may be blocked or closed at the bottom of the basket frame, to distribute the fluid uniformly along the length of the assembled tubes 54. The heating medium is therefore allowed to flow directly to the internal and other areas of the crate alleviating the problem of uneven heating.

In the exemplary embodiment illustrated by FIG. 4, the retort 20 includes a vibrating system 62 including an actuator 64 that includes a movable portion that extends into vessel 66 and is connected to a frame 66 by a linkage 68. The actuator 64 is used to apply an impulse to the frame 66 to induce motion of the frame, cube 14 and product containers relative to the vessel 67. In this embodiment, the delivery line 56 may be formed of a flexible tube to allow for movement of the cube 14.

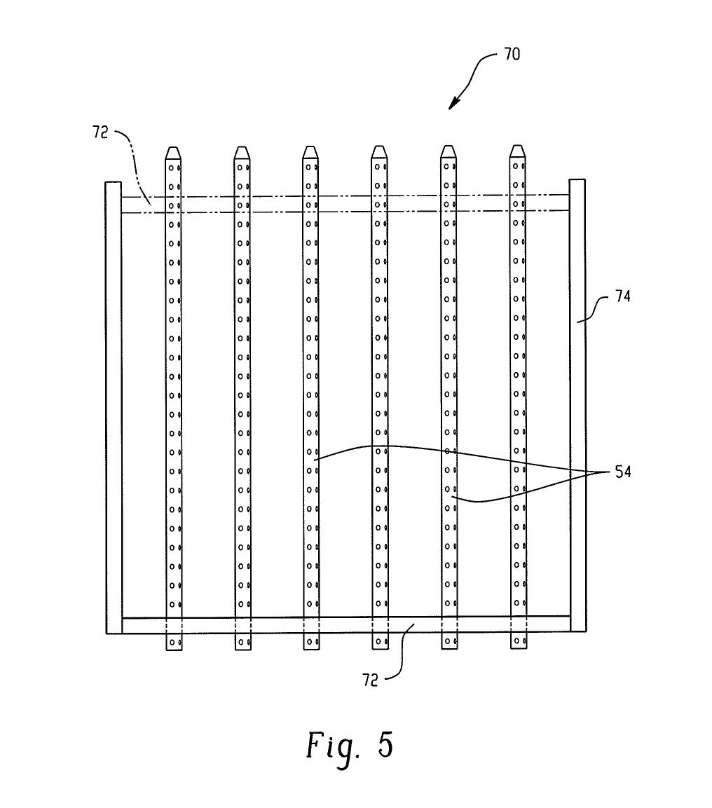

Referring to FIG. 5, an alternative embodiment 70 is designed to accommodate a basket without trays, such as where product is dumped in a container. In this system, the tubes 54 are permanently fixed to a bottom 72 of the basket 74. The bottom 72, which is raised up and down in the basket, contains cut outs for each tube 54, so that it can be raised from its resting position in the bottom of the basket, to a flush position at the top of the basket. The product can be distributed around the tubes 54 as required.

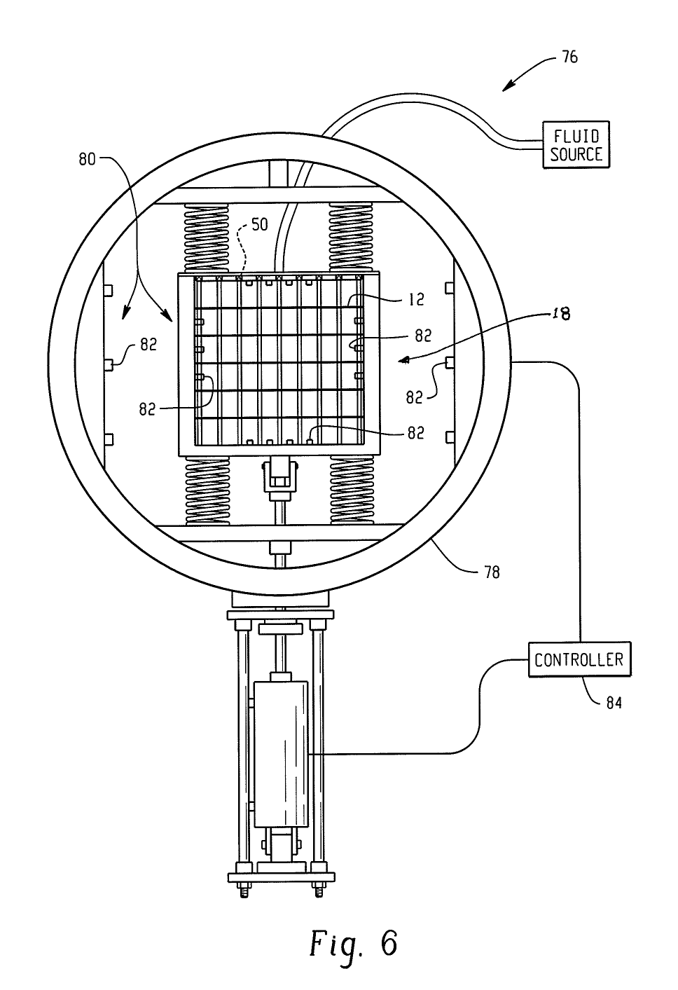

Referring to FIG. 6, a retort system 76, which, in many respects, may be similar to the retort system 20 of FIG. 4 is an immersion-type retort system. The retort system 76 also includes a system 80 for introducing vibrations to a fluid within a pressure vessel 78 that contains the product. The vibrations may be provided using transducers 82 located within the vessel 78. In some embodiments, the transducers 82 are located outside the crate receiving volume 18. In other embodiments, the transducers 82 may be located within the crate receiving volume 18. For example, the transducers may be carried by the trays 12. A controller 84 may control activation of the transducers 82.

The fluid (e.g., water) serves as a fluid transfer medium for the wave energy. In some embodiments, sonic energy (i.e., less than about 20,000 Hz) may be provided. In other embodiments, ultrasonic energy (i.e., above about 20,000 Hz) may be provided. Providing the sonic or ultrasonic energy can reduce processing time comparted to systems without the system 80.

The embodiments herein described are done so in detail for exemplary purposes only, and may be subject to many different variations in design, structure, application and operation methodology. Thus, the detailed disclosures therein should be interpreted in an illustrative, exemplary manner, and not in a limited sense.

What is claimed is:

1. A retort system, comprising:
a vessel including a crate receiving volume;
a crate located in the crate receiving volume, the crate including sides defining a product receiving volume and a plurality of tubes extending between opposite sides and through the product receiving volume, the tubes including nozzles distributed along their lengths for ejecting a heated fluid during a heating operation within the retort.

2. The retort system of claim 1 further comprising a pressurized fluid delivery system operatively connected to one or more of the plurality of tubes.

3. The retort system of claim 2, wherein the pressurized fluid delivery system provides pressurized steam and/or heated water to the plurality of tubes.

4. The retort system of claim 1, wherein the crate comprises more than two stacked trays, each tray including a plurality of tube segments extending therethrough, each tube segment having nozzles distributed along its length, wherein tube segments of adjacent trays are connected together to provide the tubes extending through and distributed throughout the product receiving volume.

5. The retort system of claim 4, wherein at least some of the tube segments include a tapered end that is inserted into tube segments stacked thereon.

6. The retort system of claim 1 further comprising products distributed about the plurality of tubes.

7. For a retort, a crate sized and configured for placement within the retort, the crate capable of holding product for a heating process within the retort, the crate comprising:
sides defining a product receiving volume;
a plurality of tubes extending between opposite sides and through the product receiving volume, the tubes including nozzles distributed along their lengths for ejecting a heated fluid during a heating operation within the retort.

8. The crate of claim 7 further comprising:
a first tray including a plurality of first tube segments extending therethrough, each first tube segment having the nozzles; and
a second tray including a plurality of second tube segments extending therethrough, each second tube segment having the nozzles;
wherein the first tube segments and the second tube segments are connected to form at least part of the tubes.

9. The crate of claim 8, wherein, the first tray is stacked upon the second tray, the first tube segments connected to the second tube segments to allow distribution of the heated fluid therethrough.

10. The crate of claim 9, wherein the second tube segments include first and second ends, the first end being tapered so as to engage an end of the first tube segments of the first tray stacked thereupon.

11. The crate of claim 7 comprising more than two stacked trays, each tray including a plurality of tube segments extending therethrough, each tube segment having an array of nozzles distributed along its length, wherein tube segments of adjacent trays are connected together to provide assembled tubes extending through and distributed throughout the product receiving volume.

12. The crate of claim 7, wherein tubes are aligned to define an imagingary line that intersects opposite sides of the crate.

13. A method of processing products in a retort system, the method comprising:
locating a crate within a crate receiving volume of a vessel; and
heating products within a product receiving volume of the crate by delivering heated fluid through a plurality of tubes extending between opposite sides of the crate and through the product receiving volume, the tubes including nozzles distributed along their lengths ejecting a heated fluid during a heating operation within the retort.

14. The method of claim 13 further comprising delivering the heated fluid from a pressurized fluid delivery system operatively connected to one or more of the plurality of tubes, wherein wherein the pressurized fluid delivery system provides pressurized steam and/or heated water to the plurality of tubes.

15. The method of claim 13 further comprising assembling the crate by stacking trays one on top of another, each tray including a plurality of tube segments extending therethrough, each tube segment having nozzles distributed along its length, wherein tube segments of adjacent trays are connected together to provide the tubes extending through and distributed throughout the product receiving volume.

16. The method of claim 15 further comprising inserting upper extended portions of tube segments of a lower tray into bottom openings of tube segment of an upper tray as the upper tray is being stacked onto the lower tray.

17. A method of thermally treating a product within a retort, the method comprising:
at least partially filling a pressure vessel of the retort with a heat transfer medium; and
providing wave energy to the heat transfer medium to heat the product, the wave energy being at least at a sonic frequency or above.

18. The method of claim 17, wherein the wave energy is at a sonic or an ultrasonic frequency.

19. The method of claim 1 further comprising providing the wave energy using one or more transducers.
